(19) United States
(12) Patent Application Publication
(10) Pub. No.: US 2015/0128958 A1
(43) Pub. Date: May 14, 2015

(54) Barrier Device to Reduce Contact and Transmission of Pubic, Upper Thigh, and Buttocks Mediated Sexually Transmitted Diseases

(71) Applicant: Chintan M. Shah, Rochester, NY (US)

(72) Inventor: Chintan M. Shah, Rochester, NY (US)

(21) Appl. No.: 14/338,986

(22) Filed: Jul. 23, 2014

Related U.S. Application Data
(60) Provisional application No. 61/903,158, filed on Nov. 12, 2013.

(51) Int. Cl. A41B 9/02; A61B 19/00

(52) U.S. Cl. CPC A41B 9/026; A61B 19/00
USPC 128/846

(57) ABSTRACT

A barrier device that can prevent infected skin in the pelvic region from making contact with a sex partner consists of a pelvic garment and a genitalia access hole. The pelvic garment contains a waist opening, a left leg opening, and a right leg opening such that a user can wear the pelvic garment similar to wearing a boxer brief. In the male version, the user inserts the penis through the genitalia access hole, which is circular in shape. In the female version, the genitalia access hole, which is oval in shape, delineates the vagina. The barrier device is made of latex and provides no inconvenience to a user during sexual intercourse. The barrier device is intended to be worn with a condom for maximum protection.

The current application claims a priority to the U.S. Provisional Patent application Ser. No. 61/903,158 filed on Nov. 12, 2013.

### FIELD OF THE INVENTION

The present invention relates generally to devices and methods for preventing and reducing the spread of sexually transmitted diseases. More specifically, the present invention is a pelvic garment worn to reduce the risk of transmitting sexually transmitted diseases that spread through infected skin-to-skin and infected skin-to-genital.

### BACKGROUND OF THE INVENTION

The number of individuals affected by sexually transmitted diseases (STD) has risen at an alarming rate in the recent past. The main reasons for this increase are poor sex education and not using recommended protection. The widely known source of protection is condoms. Condoms reduce the likelihood of pregnancy and the spread of STDs. However, it is not guaranteed that condoms protect an individual against all STDs.

Most of the known STDs are transmitted through genital-to-genital contact. However, STDs such as Herpes Simplex Virus (HSV), Gonorrhea, Chlamydia, and Human Immunodeficiency Virus (HIV) are recognized as genital-to-oral STDs. Similarly, Human Papillomavirus (HPV) warts and Syphillis are transmitted through infected genital-to-skin contact. According to the World Health Organization (WHO) data, more than one million people acquire a STD or sexually transmitted infection (STI) every day. Most importantly, a majority of STIs are present without symptoms.

It has been experimentally proven that the use of condoms is greatly effective against HIV infection. Condoms function such that they block the transmission and acquisition of STDs by preventing direct contact between genitals. Therefore, a greater level of protection is provided against STDs transmitted through direct contact of genitals. However, condoms do not guarantee protection against STDs such as HPV and HSV that transfer through infected skin. The best way to prevent the spread of STIs is to avoid contact with infected skin. Most specifically, contact with pubic areas where extended infections from genitals can occur should be prevented. Even though the need has been recognized in the industry, an effective means of protection to reduce the spread of STIs has not been disclosed.

The objective of the present invention is to address the aforementioned issues. In particular, the present invention is an apparatus that can be worn to reduce the risk of STD transmission from infected skin in the pubic/pelvic region. The present invention comes as an apparatus that can be worn by either a male or a female during sexual intercourse. However, it should be emphasized that utilizing the present invention does not guarantee to prevent the spread of STDs. The present invention, which is worn along with condoms, is intended to provide an additional level of protection to reduce the likelihood of spreading STDs through rubbing and other direct contact with infected skin in the pelvic/pubic or buttock region of the body. In particular, the present invention must always be worn with a condom and not by itself in all circumstances. The effective design of the present invention allows a user to utilize the present invention with no inconvenience during sexual intercourse.

### BRIEF DESCRIPTION OF THE DRAWINGS

FIG. 1 is a perspective view of the male configuration of the present invention.

FIG. 2 is a front view of the male configuration of the present invention.

FIG. 3 is a back view of the male configuration of the present invention.

FIG. 4 is front view of the female configuration of the present invention.

FIG. 5 is a bottom view of the female configuration of the present invention.

### DETAIL DESCRIPTIONS OF THE INVENTION

All illustrations of the drawings are for the purpose of describing selected versions of the present invention and are not intended to limit the scope of the present invention.

The present invention is a barrier device that is utilized to protect a user against Sexually Transmitted Diseases (STDs) that are transmitted through infected skin in the pelvic/pubic or buttock regions. However, it should be noted that the present invention does not guarantee to prevent the spreading of a STD. For maximum protection, the present invention needs to be worn in conjunction with a traditional condom. The present invention comprises a pelvic garment 1 and a genitalia access hole 10. The pelvic garment 1 serves as a barrier device to prevent contact of infected skin in the pelvic/pubic and buttock regions of the sexual partners. The preferred embodiment of the present invention has a male configuration and a female configuration. In the male configuration, the genitalia access hole 10 provides access for the penis. Similarly, the genitalia access hole 10 delineates the vagina in the female configuration.

The pelvic garment 1 is worn by the user in order to cover the pelvic area. The pelvic garment 1 comprises a waist opening 2, a left section 3, a right section 4, a left leg hole 7, a right leg hole 8, and a crotch section 9. The left section 3 and the right section 4 each comprise a front portion 5 and a rear portion 6 such that the front pelvic region of the user and the back pelvic region of the user are covered when the pelvic garment 1 is worn. The genitalia access hole 10 traverses into the pelvic garment 1 such that the user can conveniently utilize the genitalia access hole 10 while wearing the pelvic garment 1. The genitalia access hole 10 is positioned in between the left section 3 and the right section 4 such that the user can effectively utilize the genitalia access hole 10 for the penis or the vagina. More specifically, a male user utilizes the genitalia access hole 10 of the male configuration for the penis. Likewise, a female user utilizes the genitalia access hole 10 of the female configuration for the vagina.

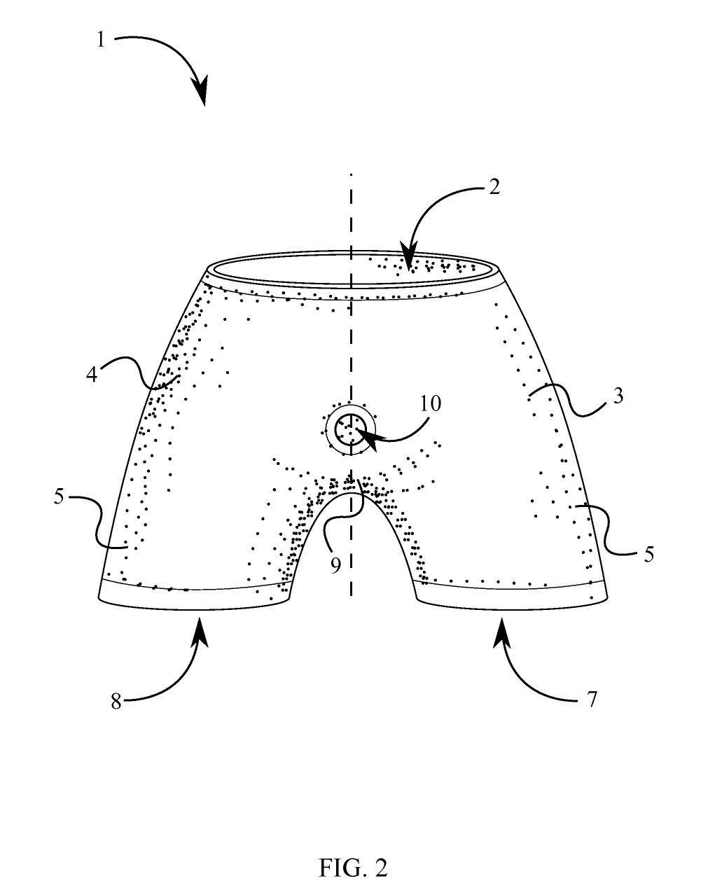

As illustrated in FIGS. 2-4, the left section 3 is positioned opposite to the right section 4 such that the pelvic garment 1 corresponds to the left side and right side of the user. The waist opening 2 is perimetrically positioned around the left section 3 and the right section 4. Moreover, the front portion 5 and the rear portion 6 which are positioned opposite to each other are perimetrically connected around the waist opening 2. The left leg hole 7 and the right leg hole 8 are positioned opposite the waist opening 2 and along the pelvic garment 1. As a result of the positioning of the waist opening 2, the left leg hole 7, and the right leg hole 8 a user can wear the pelvic garment 1 similar to wearing a boxer brief. The left leg hole 7 is perimetrically positioned around the left section 3 and allows the left leg of the user to be secured in the left leg hole 7. Similarly, the right leg hole 8 is perimetrically positioned around the right section 4 and secures the user's right leg within the right leg hole 8. As a result of wearing the pelvic garment 1, the crotch section 9, which is positioned in between the left section 3 and the right section 4, is in contact with the user's crotch.

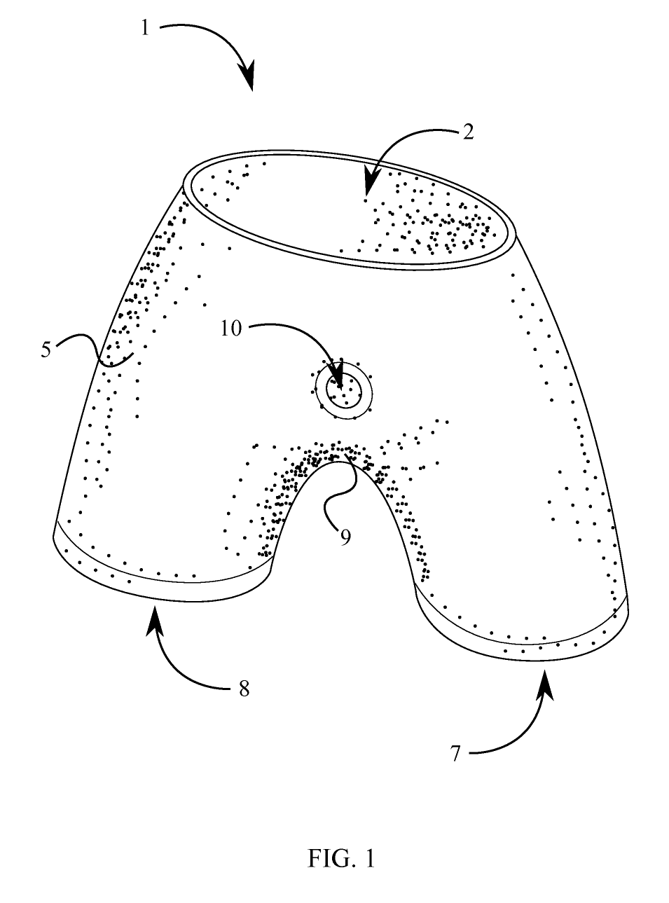

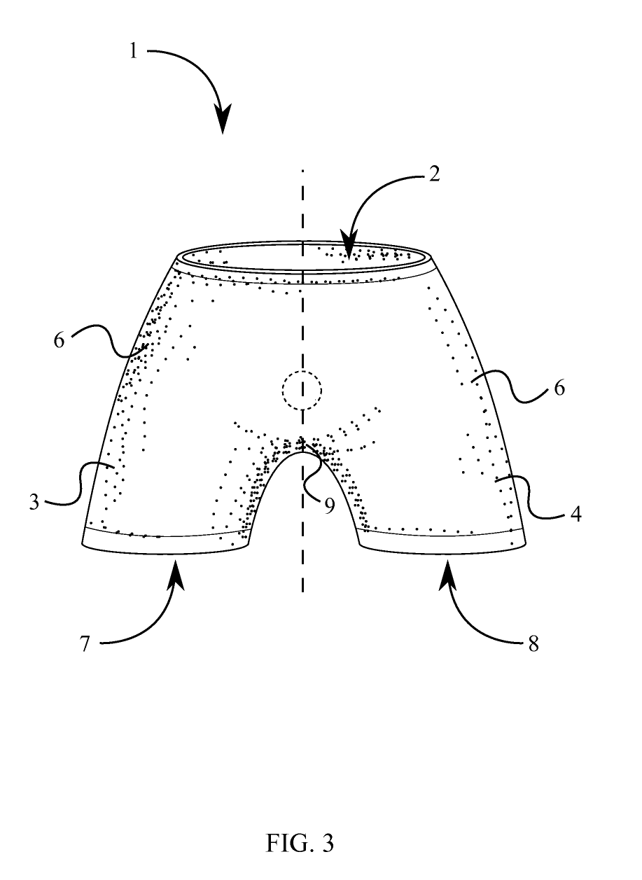

The male configuration is illustrated in FIGS. 1-3. As seen in FIG. 1 and FIG. 3, the genitalia access hole 10 traverses through the front portion 5 of both the left section 3 and the right section 4. As mentioned earlier, the genitalia access hole 10 is used to insert the penis in the male configuration. For user convenience, the genitalia access hole 10 is oriented perpendicular to the waist opening 2. In order to facilitate access for the penis, the genitalia access hole 10 has a circular shape in the male configuration.

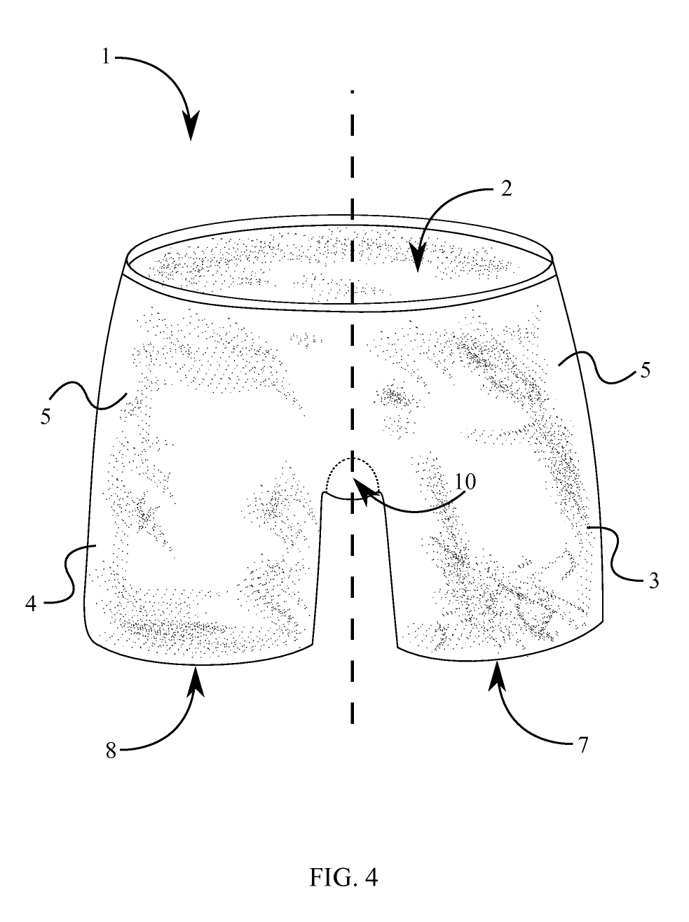

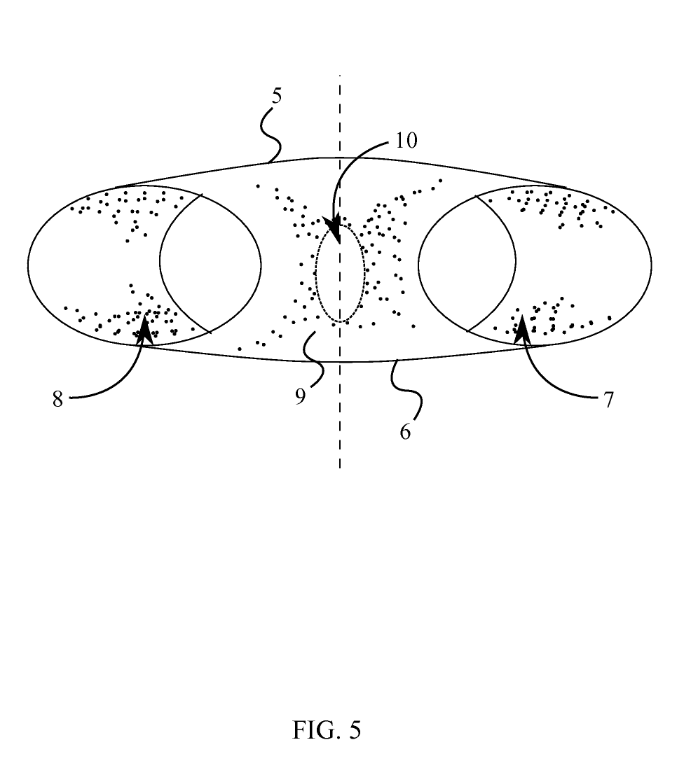

When utilizing the present invention, the female users utilize the female configuration. The female configuration is illustrated in FIG. 4 and FIG. 5. In the female configuration the genitalia access hole 10 traverses through the crotch section 9. When the female configuration is worn, the genitalia access hole 10 is utilized to delineate the vagina. In order to do so, the genitalia access hole 10 of the female configuration has an oval shape. The genitalia access hole 10 is positioned in between the left leg hole 7 and the right leg hole 8 in the female configuration. In contrast to the male configuration, the genitalia access hole 10 of the female configuration is oriented in parallel to the waist opening 2. As a result, the present invention provides no inconvenience to a female user during sexual intercourse.

Conventional condoms are made of materials that can include, but are not limited to latex, polyurethane or polyisoprene. It has been experimentally proven that condoms provide positive in reducing pregnancy and spreading STDs. The present invention which is used simultaneously with a traditional male condom or a female condom is also made of latex or any other comparable material. As a result, genital-to-genital contact, infected skin-to-skin contact, and infected skin-to-genital contact are all prevented. However, wearing the present invention along with a traditional condom does not guarantee complete protection against STDs.

In an additional embodiment of the present invention, the front portion 5 and the rear portion 6 can be separated. In the additional embodiment, the front portion 5 is a membrane. Furthermore, the front portion 5 is detachably attached to the rear portion 6. In order to attach the front portion 5 to the rear portion 6, an attachment mechanism is utilized in between the front portion 5 and the rear portion 6. The attachment mechanism can be, but is not limited to, a string attachment mechanism and a hook and loop attachment mechanism. However, the additional embodiment can also be utilized with the absence of the rear portion 6. In such circumstances, the attachment mechanism is utilized to attach the membrane to the user's body.

If the string attachment mechanism is utilized, an upper pair of strings and a lower pair of strings is utilized to fasten the front portion 5 to the rear portion 6 of the additional embodiment. In the absence of a rear portion, the upper pair of strings secures the front portion, which is the membrane, around the user's waist. The lower pair of strings secures the membrane around the thigh regions of the user.

If the hook and loop attachment mechanism is utilized in the additional embodiment, the hook and loop attachment mechanism comprises a pair of waist bands, a pair of left leg bands, and a pair of right leg bands. In the absence of the rear portion 6, the pair of waist bands secures the membrane around the waist by engaging a hook portion of the hook and loop attachment mechanism with a loop portion of the hook and loop attachment mechanism. The pair of left leg bands secures the membrane with the left leg of the user by engaging the hook portion with the loop portion. Similarly, the right leg band secures the membrane with the right leg of the user by engaging the hook portion with the loop portion. As a result, the front portion 5 is secured against the front pelvic region of the user. Similar to the preferred embodiment of the present invention, the additional embodiment also includes a male configuration and a female configuration.

In utilizing the present invention, the consequent process flow is followed. Initially, if the intended user is a male, the male configuration is chosen. Likewise, if the intended user is a female, the female configuration is chosen. Next, the user wears the pelvic garment 1 similar to wearing a boxer brief. As a result, the waist opening 2 will be secured around the waist. The left leg hole 7 and the right leg hole 8 will also be simultaneously secured around the respective legs. Additionally, the circular shaped genitalia access hole 10 will be positioned to provide access to the penis in the male configuration. Similarly, the oval shaped genitalia access hole 10 will delineate the vagina in the female configuration.

Although the invention has been explained in relation to its preferred embodiment, it is to be understood that many other possible modifications and variations can be made without departing from the spirit and scope of the invention as hereinafter claimed.

What is claimed is:

1. A barrier device to reduce contact and transmission of pubic, upper thigh, and buttocks mediated sexually transmitted diseases comprises:
a pelvic garment;
a genitalia access hole;
the pelvic garment comprises a waist opening, a left section, a right section, a left leg hole, a right leg hole, and a crotch section;
the left section and the right section each comprise a front portion and a rear portion;
the genitalia access hole traversing into the pelvic garment; and
the genitalia access hole being positioned in between the left section and the right section.

2. The barrier device to reduce contact and transmission of pubic, upper thigh, and buttocks mediated sexually transmitted diseases as claimed in claim 1 comprises:
the left section being oppositely positioned from the right section;
the waist opening being perimetrically positioned around the left section and the right section;
the left leg hole and the right leg hole being oppositely positioned from the waist opening along the pelvic garment;
the left leg hole being perimetrically positioned around the left section;
the right leg hole being perimetrically positioned around the right section;
the crotch section being positioned in between the left section and the right section;
the front portion and the rear portion being perimetrically connected around the waist opening; and
the front portion being positioned opposite of the rear portion.

3. The barrier device to reduce contact and transmission of pubic, upper thigh, and buttocks mediated sexually transmitted diseases as claimed in claim 1 comprises:
the genitalia access hole traversing through the front portion of both the left section and the right section, wherein the genitalia access hole is used to insert a penis; and
the genitalia access hole being oriented perpendicular to the waist opening.

4. The barrier device to reduce contact and transmission of pubic, upper thigh, and buttocks mediated sexually transmitted diseases as claimed in claim 3, wherein the genitalia access hole being a circular shape.

5. The barrier device to reduce contact and transmission of pubic, upper thigh, and buttocks mediated sexually transmitted diseases as claimed in claim 1 comprises:
the genitalia access hole traversing through crotch section, wherein the genitalia access hole is used to delineate a vagina;
the genitalia access hole being positioned in between the left leg hole and the right leg hole; and
the genitalia access hole being oriented parallel to the waist opening.

6. The barrier device to reduce contact and transmission of pubic, upper thigh, and buttocks mediated sexually transmitted diseases as claimed in claim 5, wherein the genitalia access hole being an oval shape.

7. The barrier device to reduce contact and transmission of sexually transmitted diseases as claimed in claim 1, wherein the pelvic garment is made of a latex material.

8. A barrier device to reduce contact and transmission of pubic, upper thigh, and buttocks mediated sexually transmitted diseases comprises:
a pelvic garment;
a genitalia access hole;
the pelvic garment comprises a waist opening, a left section, a right section, a left leg hole, a right leg hole, and a crotch section;
the left section and the right section each comprise a front portion and a rear portion;
the genitalia access hole traversing into the pelvic garment;
the genitalia access hole being positioned in between the left section and the right section;
the left section being oppositely positioned from the right section;
the waist opening being perimetrically positioned around the left section and the right section;
the left leg hole and the right leg hole being oppositely positioned from the waist opening along the pelvic garment;
the left leg hole being perimetrically positioned around the left section;
the right leg hole being perimetrically positioned around the right section;
the crotch section being positioned in between the left section and the right section;
the front portion and the rear portion being perimetrically connected around the waist opening;
the front portion being positioned opposite of the rear portion; and
the pelvic garment being made of latex.

9. The barrier device to reduce contact and transmission of pubic, upper thigh, and buttocks mediated sexually transmitted diseases as claimed in claim 8 comprises:
the genitalia access hole traversing through the front portion of both the left section and the right section, wherein the genitalia access hole is used to insert a penis;
the genitalia access hole being oriented perpendicular to the waist opening; and
the genitalia access hole being circular.

10. The barrier device to reduce contact and transmission of pubic, upper thigh, and buttocks mediated sexually transmitted diseases as claimed in claim 8 comprises:
the genitalia access hole traversing through crotch section, wherein the genitalia access hole is used to delineate a vagina;
the genitalia access hole being positioned in between the left leg hole and the right leg hole;
the genitalia access hole being oriented parallel to the waist opening; and
the genitalia access hole being oval.
